(19) United States
(12) Patent Application Publication
(10) Pub. No.: US 2024/0287458 A1
(43) Pub. Date: Aug. 29, 2024

(54) METHOD OF OBTAINING HSC POPULATION, T CELL POPULATION AND NK CELL POPULATION AND COMPOSITIONS THEREOF

(71) Applicant: Regrow Biosciences Private Limited, Mumbai (IN)

(72) Inventors: Satyen Yeshwant Sanghavi, Mumbai (IN); Vinayak Virupaksh Kedage, Lonavala (IN)

(21) Appl. No.: 18/571,907

(22) PCT Filed: Aug. 16, 2021
(86) PCT No.: PCT/IN2021/050785

§ 371 Date: Dec. 19, 2023

(30) Foreign Application Priority Data
Jun. 30, 2021 (IN) 202121029375

(51) Int. Cl. C12N 5/0789; C12N 5/00; C12N 5/0783

(52) U.S. Cl. CPC C12N 5/0647; C12N 5/0087; C12N 5/0636; C12N 5/0646; C12N 2501/50; C12N 2506/025

(57) ABSTRACT

The present disclosure discloses pharmaceutical compositions comprising hematopoietic stem cells (HSC) cells, NK cells, and T cells individually, having positive and negative surface markers and pharmaceutically acceptable excipients. The present disclosure further relates to the method for preparing the composition thereof. The present disclosure also provides a method of treating a subject comprising the use of any of said compositions in treating diseases selected from the group consisting of graft versus host disease, malignant disease, and non-malignant disease.

### FIELD OF THE INVENTION

The present disclosure broadly relates to the field of development, cell biology, molecular biology, genetics and stem-cell research. In particular, the present disclosure relates to a method of preparing enriching hematopoetic stem cells, Natural Killer cells and T cells, compositions and implications thereof.

### BACKGROUND OF THE INVENTION

Stem cells are primal cells found in most multi-cellular organisms and known for their self-renewing characteristic that helps in continuous replenishing of cells and tissues of the body. Human stem cells are precursor cells capable of generating a variety of mature human cell lineages. Further, the stem cells possessing capability of reconstituting various cell lineages, can be obtained from various sources including, bone marrow (BM), mobilized peripheral blood (MPB), fetal liver, placenta, embryonic stem cells, and umbilical cord blood (UCB), however, the limitation of expansion of isolated stem cells acts as restriction for their further use in therapeutic purposes.

Among the many types of stem cells, hematopoietic stem cells (HSC) are one of the most widely studied cells because of their capability to reproduce and differentiate into all kinds of blood cells, including erythroid, myeloid, and lymphoid lineages. The erythroid lineage comprises the red blood cells, the myeloid lineage comprises of monocytes, granulocytes, megakaryocytes, platelets, cells monitoring the presence of foreign bodies or material in the blood stream; and the lymphoid lineage comprises of B and T cells. Thus, HSCs have a high therapeutic potential to remedy high-risk hematological malignancies, as well as other diseases of blood-forming cells and the immune system including cancers, leukemia, lymphoma, cardiac failure, neural disorders, auto-immune diseases, immunodeficiency, metabolic or genetic disorders. Stem cell-based therapies aim to heal or replace diseased/destroyed cells or organs or body parts with healthy new cells provided by stem cell transplantation.

Natural killer (NK) cells are essential effector cells of the innate immune system. The NK cells rapidly recognize and kills virally infected and transformed cells, mediate inflammation, and regulate innate and adaptive immune function via interaction with other immune cells by killing infected and transformed cells, inflammation, engraftment of hematopoetic stem cells and regulation of immune function. NK cells represent one of the first lines of host immune defense and have been used to treat viral infections and malignancies as well as to increase engraftment of bone marrow stem cell transplants in experimental animal models and humans.

As part of adaptive immunity, T cells play an integral role in executing and controlling immune responses. T cells can be distinguished from other lymphocytes, such as B cells and natural killer (NK) cells, by the presence of a T cell receptor on the cell surface. Each of the various T cell subsets has distinct functions and aids the immune response to achieve best efficacy. Upon antigen encounter, T cells are activated, differentiate into a specific subtype, expand, and fulfill their role as effector cells, e.g., by migrating into various tissues and organs.

The explicit self-renewing feature of hematopoietic stem cell to replenish different blood cells throughout the life-time makes it a considerable area of interest for therapeutic studies. Extensive clinical research for many years has lead to various methods and techniques for the identification, isolation and expansion of human hematopoetic stem cells. For instance, U.S. Pat. No. 8,057,789B2 relates to the compositions and methods of using placental stem cells originating from a postpartum placenta with conventional cord blood compositions or other stem or progenitor cells. The patent further relates to the use of placental stem cell and mixed population for the treatment of diseases or disorders.

Patent application WO2018197868A1 relates to the method for expansion of hematopoietic stem and progenitor stem cells (HSPC) population and a kit comprising sterile elements for the expansion of HSPC, a HDAC inhibitor and an aminothiol compound or a pharmaceutically acceptable salt thereof.

U.S. Pat. No. 8,506,955B2 also relates to an in-vitro method of propagating hematopoietic stem cells, which comprises culturing the cells in medium with one or more angiopoietin-like proteins, under conditions sufficient for expansion of HSCs. The patent further relates to a method of administering hematopoietic stem cells to an individual for therapeutic purposes.

Patent CN107206100B broadly relates to compositions and methods for stimulating and producing or expanding natural killer (NK) cells. The patent also describes methods of treating cancer using the disclosed NK-stimulating exosomes or NK cells stimulated with the disclosed methods.

Despite of the extensive research for the use of hematopoetic stem cells (HSCs) in the treatment of various diseases and disorders, the use of HSCs transplantation remains limited because of the lack of HSCs sources and complexities in the process of expansion of cells for therapeutic needs. The existing strategies for isolation and expansion of HSC's, Natural killer cells, and T cells are still compromising either with the cell count or cell purification aspects. Severe graft-versus-host disease (GVHD), human leukocyte antigen (HLA) matching, viral transmission to donors, graft-failure, affordability and availability are some more restricting factors governing the use of hematopoietic stem cell populations. Further, the purpose-specific enrichment of HSC's, Natural killer cells, and T cells have become an equally important aspect along with the isolation and purification for their further use.

The afore-mentioned limitations associated with the isolation and purification of the HSC's have greatly hampered their clinical utility. Therefore, there still exist a need in the present field of art to develop an efficient and affordable method for the production of pure and enriched hematopoietic stem cell population.

### SUMMARY OF THE INVENTION

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

In an aspect of the present disclosure, there is provided a method for obtaining enriched haemopoietic stem cells, natural killer cells and T cells, wherein the method comprising: (a) separating leucocytes from umbilical cord blood using a sedimentation reagent to obtain leucocyte rich plasma, wherein the sedimentation buffer comprises at least one of the reagents selected from the group consisting of hydroxy ethyl starch, gelatin, chitosan, and combinations thereof; (b) adding red blood cell lysis buffer to the leucocyte rich plasma to obtain leucocyte concentrate, wherein the red blood cell lysis buffer comprises ammonium chloride and a protein combination comprising at least one protein derived from umbilical cord blood and maternal blood, wherein the leucocyte concentrate is free of red blood cells; (c) incubating the leucocyte concentrate with CD34⁺, CD133⁺ and CD38⁻ immunomagnetic beads for obtaining hematopoietic stem cells; (d) incubating the leucocyte concentrate with CD56⁺ and CD3⁻ immunomagnetic beads for obtaining natural killer cells; and (e) incubating the leucocyte concentrate with CD3+ immunomagnetic beads for obtaining T cells.

In another aspect of the present disclosure, there is provided an enriched hematopoietic stem cells (HSC) obtained by the method as described herein.

In another aspect of the present disclosure, there is provided an enriched natural killer cells obtained by the method as described herein.

In another aspect of the present disclosure, there is provided an enriched T cells obtained by the method as described herein.

In yet another aspect of the present disclosure, there is provided a composition comprising: (a) an enriched hematopoietic stem cells (HSC) having positive and negative surface markers; and (b) pharmaceutically acceptable excipients selected from HSA, DMEM or combinations thereof.

In yet another aspect of the present disclosure, there is provided a composition comprising: (a) an enriched natural killer cells, wherein natural killer cells are having positive and negative surface markers; and (b) pharmaceutically acceptable excipients selected from HSA, DMEM or combinations thereof.

In still another aspect of the present disclosure, there is provided a composition comprising: (a) an enriched T cells wherein T cells are having positive and negative surface markers; and (b) pharmaceutically acceptable excipients selected from HSA, DMEM or combinations thereof.

In yet another aspect of the present disclosure, there is provided an isolated cell population enriched for CD34+, CD133+ and CD38− haematopoietic stem cells.

In yet another aspect of the present disclosure, there is provided an isolated cell population enriched for CD56⁺, and CD3⁻ Natural killer cells.

In yet another aspect of the present disclosure, there is provided an isolated cell population enriched for CD3+ T cells.

In an alternate aspect of the present disclosure, there is provided a method of treating diseases selected from the group consisting of graft versus host disease, malignant disease, and non-malignant disease, said method comprising administering a therapeutic amount of the composition in a subject as described herein.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts the images of fluorescence-activated cell sorting (FACS) results as obtained in case of HSC's enrichment after RBC depletion with Method-I (ammonium chloride lysis buffer) performed in Example 4, in accordance with an embodiment of the present disclosure.

FIG. 2 depicts the images of fluorescence-activated cell sorting (FACS) results as obtained in case of HSC's enrichment after RBC depletion with Method-II (ammonium chloride lysis buffer and feotal bovine serum (FBS) combination) performed in Example 4, in accordance with an embodiment of the present disclosure.

FIG. 3 depicts the images of fluorescence-activated cell sorting (FACS) results as obtained in case of HSC's enrichment after RBC depletion with Method-III (ammonium chloride lysis buffer and human serum albumin (HSA) combination) performed in Example 4, in accordance with an embodiment of the present disclosure.

FIG. 4 depicts the images of fluorescence-activated cell sorting (FACS) results as obtained in case of HSC's enrichment after RBC depletion with Method-IV (ammonium chloride lysis buffer and blood plasma derived protein combination) performed in Example 4, in accordance with an embodiment of the present disclosure.

FIG. 5 depicts the images of fluorescence-activated cell sorting (FACS) results as obtained in case of NK cell enrichment after RBC depletion with Method-I (ammonium chloride lysis buffer) performed in Example 4, in accordance with an embodiment of the present disclosure.

FIG. 6 depicts the images of fluorescence-activated cell sorting (FACS) results as obtained in case of NK cell enrichment after RBC depletion with Method-II (ammonium chloride lysis buffer and feotal bovine serum (FBS) combination) performed in Example 4, in accordance with an embodiment of the present disclosure.

FIG. 7 depicts the images of fluorescence-activated cell sorting (FACS) results as obtained in case of NK cell enrichment after RBC depletion with Method-III (ammonium chloride lysis buffer and human serum albumin (HSA) combination) performed in Example 4, in accordance with an embodiment of the present disclosure.

FIG. 8 depicts the images of fluorescence-activated cell sorting (FACS) results as obtained in case of NK cell enrichment after RBC depletion with Method-IV (ammonium chloride lysis buffer and blood plasma derived protein combination) performed in Example 4, in accordance with an embodiment of the present disclosure.

FIG. 9 depicts the images of fluorescence-activated cell sorting (FACS) results as obtained in case of T cell enrichment after RBC depletion with Method-I (ammonium chloride lysis buffer) performed in Example 4, in accordance with an embodiment of the present disclosure.

FIG. 10 depicts the images of fluorescence-activated cell sorting (FACS) results as obtained in case of T cell enrichment after RBC depletion with Method-II (ammonium chlorid0e lysis buffer and feotal bovine serum (FBS) combination) performed in Example 4, in accordance with an embodiment of the present disclosure.

FIG. 11 depicts the images of fluorescence-activated cell sorting (FACS) results as obtained in case of T cell enrichment after RBC depletion with Method-III (ammonium chloride lysis buffer and human serum albumin (HSA) combination) performed in Example 4, in accordance with an embodiment of the present disclosure.

FIG. 12 depicts the images of fluorescence-activated cell sorting (FACS) results as obtained in case of T cell enrichment after RBC depletion with Method-IV (ammonium chloride lysis buffer and blood plasma derived protein combination) performed in Example 4, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “umbilical cord blood” (UCB) as used herein refers to the blood that remains in the placenta and in the attached umbilical cord after childbirth. Cord blood is a a valuable source of stem cells, which can be used to treat hematopoietic and genetic disorders. The terms, “umbilical cord blood”, “cord blood”, “UCB”, “CB” are interchangeably used throughout the draft and relates to the same meaning as described herein.

The term “maternal blood” (MB) as used herein refers to the blood collected from a mother pre- and post-delivery. The maternal blood (MB) collection may take place at a time immediately before/after cord blood collection, at the time of admission for delivery (after initiation of labour) or before transfusion/infusion of any intravenous fluid (colloids/crystalloids/blood products). The terms, “maternal blood”, “MB” are interchangeably used throughout the draft and relates to the same meaning as described herein.

The term “umbilical cord blood and maternal blood” (UCB+MB) as used herein means any combination of an umbilical cord blood and maternal blood. Said combination may come from autologous and/or allogenic sources.

The term “hematopoietic stem cell” (HSC) relates to the cell isolated from the blood or bone marrow and possess to ability to renew itself, to differentiate to a variety of specialized cells, to mobilize out of the bone marrow into circulating blood, to undergo programmed cell death. The plural form is also referred as hematopoietic stem cells (HSC's).

The term “Natural killer cells”, also known as “NK cells” or large granular lymphocytes (LGL), relates to a type of cytotoxic lymphocyte critical to the innate immune system that belong to the rapidly expanding family of innate lymphoid cells (ILC) and represent 5-20% of all circulating lymphocytes in humans. The role of NK cells is analogous to that of cytotoxic T cells in the vertebrate adaptive immune response. NK cells can be identified by the presence of CD56 and the absence of CD3 (CD56+, CD3−).

The term “T cells” relates to a type of lymphocyte that belongs to one of the important white blood cells of the immune system, which play a central role in the adaptive immune response. T cells are borne from hematopoietic stem cells and can be easily be distinguished from other lymphocytes by the presence of a T-cell receptor (TCR) on their cell surface.

The term “enrichment” refers to the method of improving/increasing the proportion of a hematopoietic stem cells having a particular surface marker in a HSC population. As per the disclosure made herein, the isolated cell population was further enriched for the CD34+, CD56+, and CD3+ cell surface marker.

The term “Leucocyte rich plasma” refers to the is a concentrate of leucocyte-rich plasma protein derived from whole blood, centrifuged to remove red blood cells. It is obtained by sedimentation of RBCs using sedimentation reagent, collection of plasma and centrifugation and after that excess plasma is removed and the final solution remained with some amount of plasma with concentrated cells is called as “Leucocyte Rich Plasma”

The term free of red blood cells defines the blood without the red blood cells. In other words, free of red blood cells means absence/devoid of Red blood cells obtained by lysis of RBCs.

The term “blood-derived proteins” refers to the group of proteins derived from the host's blood sample. Further, the terms “cord blood derived proteins” and “maternal blood-derived proteins” refers to the proteins extracted from the umbilical cord blood or cord blood sample and maternal blood sample respectively. As per the present disclosure, the combination of cord blood and maternal blood proteins were derived from the combination of cord blood and maternal blood mixed in a ratio of 10:1.

The term “pharmaceutical acceptable excipients” as used herein refers to an inactive substance formulated alongside the active ingredient (“API”) of a medication, for the purpose of bulking-up formulations that contain potent active ingredients, thus often referred to as “bulking agents,” “fillers,” or “diluents”. The pharmaceutical acceptable excipients in accordance with the present disclosure may be selected but not limited to the group including HSA, DMEM or combinations thereof.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

Hematopoietic stem cell transplantation (HSCT) has become the standard of care for many patients with defined congenital or acquired disorders of the hematopoietic system or with chemosensitive, radiosensitive, or immunosensitive malignancies (Gratwohl A, Baldomero H, Aljurf M, et al. Hematopoietic Stem Cell Transplantation: A Global Perspective. JAMA. 2010; 303(16):1617-1624. doi:10.1001/jama.2010.491). However, the limitations over the availability and affordability of the hematopoietic stem cells is continuously hampering the extent of their practical use for therapeutic purposes.

As explained in the literature, various efforts have been made in the existing methods for ex vivo expansion of human cord blood hematopoietic stem cells (HSCs) and hematopoietic progenitor cells (HPCs), in order to acquire a larger number of transplantable HSCs and HPC's. However, there still are evidences of dearth in the coventional methods in terms of achieving higher cell expansion capacity, higher recovery of surface marker-specific cell population, higher cell viability, and minimizing cell apoptosis in the expanded cell population. Also, the methods of HSC's isolation and enrichment generally compromised either with the cell count or cell purification aspects depending upon the purpose-specific use.

The use of HSCs transplantation remains limited by the lack of HSC's sources and complexities in the process of expansion of cells for therapeutic needs, whereas the method as disclosed herein relates to the isolation and enrichment of HSC's from umbilical cord blood (UCB) samples, thereby facilitating rapid and convenient availability from numerous cord blood banks, less stringent criteria for human leukocyte antigen (HLA) matching, lower chances of severe graft-versus-host disease (GVHD) without compromising graft-versus-leukemia effects, lower risk of viral transmission and the absence of risk to donors. The method involves double-sedimentation step for the isolation of leucocyte concentrate from human umbilical cord blood, followed by RBC lysis and further marker-specific cell enrichment. The marker-specific cell population is subjected to ex-vivo cell expansion for use in allogenic hematopoietic stem cell transplant or other therapeutic purposes. The immunomagnetic cell separation method employed in the disclosed method facilitates the positive selection of CD34⁺, CD133⁺ and CD38⁻ hematopoetic stem cells, CD56⁺, and CD3⁻ NK cells and CD3⁺ T cells. The RBC's reduction and maximum recovery of hematopoietic stem cells or required cells in the present method was achieved by double sedimentation method using sedimentation reagent such as hydroxyethyl starch (HES), gelatin and chitosan alone or in combination. Chitosan being positively charged due to free amino groups resulting from chitin deacetylation are protonated at physiological pH, facilitates appreciable RBCs depletion and HSCs recovery from the umbilical cord blood on aggregation with negatively charged erythrocytes. Starch and gelatin also provides enhanced sedimentation of unwanted blood cells.

Further, the present method involves the use of cord and maternal plasma proteins combination in addition to ammonium chloride for RBCs lysis, which showed protective effect on cells viability even at higher concentration of ammonium chloride used for the said purpose. The cord and maternal plasma proteins combination employed in the method disclosed herein also found effective over the free-radical and oxidative stress generated by ammonium chloride on targeted HSCs, NK and T cell population, thereby maximizing the target cell recovery. The immunomagnetic cell separation step helps in further depletion of unwanted cells for in vitro expansion of target cells.

In an aspect of the present disclosure, there is provided a method for obtaining enriched haemopoietic stem cells, natural killer cells and T cells, wherein the method comprising: (a) separating leucocytes from umbilical cord blood using a sedimentation reagent to obtain leucocyte rich plasma, wherein the sedimentation buffer comprises at least one of the reagents selected from the group consisting of hydroxy ethyl starch, gelatin, chitosan, and combinations thereof; (b) adding red blood cell lysis buffer to the leucocyte rich plasma to obtain leucocyte concentrate, wherein the red blood cell lysis buffer comprises ammonium chloride and a protein combination comprising at least one protein derived from umbilical cord blood and maternal blood, wherein the leucocyte concentrate is free of red blood cells; (c) incubating the leucocyte concentrate with CD34⁺, CD133⁺ and CD38⁻ immunomagnetic beads for obtaining hematopoietic stem cells; (d) incubating the leucocyte concentrate with CD56⁺ and CD3⁻ immunomagnetic beads for obtaining natural killer cells; and (e) incubating the leucocyte concentrate with CD3⁺ immunomagnetic beads for obtaining T cells.

In another aspect of the present disclosure, there is provided a method as described here, wherein the hydroxy ethyl starch is in the concentration range of 4-12% with the molecular weight in the range of 130 kDa-500 kDa, the gelatin is in the concentration range of 3-6% with the molecular weight in the range of 30-60 kDa, and the chitosan with the molecular weight in the range of 50-247 kDa.

In another aspect of the present disclosure, there is provided a method as described, wherein the red blood cell lysis buffer is obtained by a method comprising: (a) obtaining a plasma from an umbilical cord blood; (b) obtaining a plasma from a maternal blood; (c) mixing the plasma from umbilical cord blood and the plasma from maternal blood to obtain a first mixture; adding saturated ammonium sulphate to the first mixture in ratio of 1:1 followed by centrifugation at 3000-5000 rpm for 5 minutes to obtain the protein precipitate in the form of pellet; (d) suspending the pellet in water followed by centrifugation at 11,000-14,000 rpm to obtain an enriched plasma; and (e) adding ammonium chloride to the enriched plasma to obtain the red blood cell lysis buffer at pH 7.2, wherein said lysis buffer comprises ammonium chloride and protein combinations, wherein said protein combination comprises antioxidative protein, serine/threonine protein kinase, transmembrane protein and heat shock proteins.

In another aspect of the present disclosure, there is provided a method as described, wherein the red blood cell lysis buffer is obtained by a method comprising: (a) obtaining a plasma from an umbilical cord blood; (b) obtaining a plasma from a maternal blood; (c) mixing the plasma from umbilical cord blood and the plasma from maternal blood to obtain a first mixture; adding saturated ammonium sulphate to the first mixture in ratio of 1:1 followed by centrifugation at 3000-5000 rpm for 5 minutes to obtain the protein precipitate in the form of pellet; (d) suspending the pellet in water followed by centrifugation at 11,000-14,000 rpm to obtain an enriched plasma; and (e) adding ammonium chloride to the enriched plasma to obtain the red blood cell lysis buffer (pH 7.2), wherein said lysis buffer comprises ammonium chloride and a protein combination, wherein said protein combination comprises antioxidative protein, serine/threonine protein kinase, transmembrane protein and heat shock proteins, and wherein the antioxidative protein is peroxiredoxin-1 protein and heat shock proteins are heat shock cognate 71 kDa protein isoform 2.

In an aspect of the present disclosure, there is provided a method for obtaining enriched haemopoietic stem cells, natural killer cells and T cells, wherein the method comprising: (a) separating leucocytes from umbilical cord blood using a sedimentation reagent to obtain leucocyte rich plasma, wherein the sedimentation buffer comprises at least one of the reagents selected from the group consisting of hydroxy ethyl starch, gelatin, chitosan, and combinations thereof; (b) adding red blood cell lysis buffer to the leucocyte rich plasma to obtain leucocyte concentrate, wherein the red blood cell lysis buffer comprises ammonium chloride and a protein combination comprising at least one protein derived from umbilical cord blood and maternal blood, wherein the leucocyte concentrate is free of red blood cells; (c) incubating the leucocyte concentrate with CD34+, CD133+ and CD38− immunomagnetic beads for obtaining hematopoietic stem cells; (d) incubating the leucocyte concentrate with CD56+ and CD3− immunomagnetic beads for obtaining natural killer cells; and (e) incubating the leucocyte concentrate with CD3+ immunomagnetic beads for obtaining T cells and, wherein the hematopoietic stem cells, natural killer cells, and T cells have a purity of at least 90% and are obtained in the range of 10⁵-10⁶ cells.

In an aspect of the present disclosure, there is provided a method for obtaining enriched haemopoietic stem cells, natural killer cells and T cells, wherein the method comprising: (a) separating leucocytes from umbilical cord blood using a sedimentation reagent to obtain leucocyte rich plasma, wherein the sedimentation buffer comprises at least one of the reagents selected from the group consisting of hydroxy ethyl starch, gelatin, chitosan, and combinations thereof; (b) adding red blood cell lysis buffer to the leucocyte rich plasma to obtain leucocyte concentrate, wherein the red blood cell lysis buffer comprises ammonium chloride and a protein combination comprising at least one protein derived from umbilical cord blood and maternal blood, wherein the leucocyte concentrate is free of red blood cells; (c) incubating the leucocyte concentrate with CD34+, CD133+ and CD38− immunomagnetic beads for obtaining hematopoietic stem cells; (d) incubating the leucocyte concentrate with CD56+ and CD3− immunomagnetic beads for obtaining natural killer cells; and (e) incubating the leucocyte concentrate with CD3+ immunomagnetic beads for obtaining T cells and, wherein the method provides an enriched hematopoietic stem cells (HSC), enriched natural killer cells and an enriched T cells.

In yet another aspect of the present disclosure, there is provided a composition comprising: (a) an enriched hematopoietic stem cells (HSC) having positive and negative surface markers; and (b) pharmaceutically acceptable excipients selected from HSA, DMEM or combinations thereof.

In yet another aspect of the present disclosure, there is provided a composition comprising: (a) an enriched hematopoietic stem cells (HSC) having positive and negative surface markers; and (b) pharmaceutically acceptable excipients selected from HSA, DMEM or combinations thereof, wherein said positive surface markers for hematopoietic stem cells are selected from the group consisting of CD34+, CD133+ and CD38−, and the negative markers selected from the group consisting of CD2, CD3, CD1b, CD14, CD15, CD16, CD19, CD56, CD123, and CD235a.

In yet another aspect of the present disclosure, there is provided a composition comprising: (a) an enriched natural killer cells wherein natural killer cells are having positive and negative surface markers; and (b) pharmaceutically acceptable excipients selected from HSA, DMEM or combinations thereof, wherein said positive surface markers for natural killer cells are selected from the group consisting of CD56⁺, and CD3⁻ and negative markers selected from the group consisting of CD34, and CD19.

In still another aspect of the present disclosure, there is provided a composition comprising: (a) an enriched T cells wherein T cells are having positive and negative surface markers; and (b) pharmaceutically acceptable excipients selected from HSA, DMEM or combinations thereof, and wherein said positive surface markers for T cells is CD3+ and said negative markers are selected from CD56, CD34, and CD19.

In yet another aspect of the present disclosure, there is provided a therapeutical use of the composition as described herein in a subject.

In yet another aspect of the present disclosure, there is provided an isolated cell population enriched for CD34+, CD133+ and CD38− haematopoietic stem cells.

In yet another aspect of the present disclosure, there is provided an isolated cell population enriched for CD56⁺, and CD3⁻ Natural killer cells.

In yet another aspect of the present disclosure, there is provided an isolated cell population enriched for CD3+ T cells.

In an alternate aspect of the present disclosure, there is provided a method of treating diseases selected from the group consisting of graft versus host disease, malignant disease, and non-malignant disease, said method comprising administering a therapeutic amount of the composition in a subject as described herein.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

### Example 1

### Umbilical Cord Blood Collection

The present method involves the use of human umbilical cord blood as a source of hematopoietic stem cells (HSCs) for marker-specific enrichment and expansion for further therapeutic uses. The volunteers for the cord blood donation were asked to sign informed consent forms prior to collection. The present study was performed on umbilical cord blood (UCB) samples that were obtained from normal full-term deliveries as per the standard method. All individuals with known history of infectious diseases, diabetes mellitus, severe hypertension, abortions or bad obstetric history were excluded from the study.

As per the present disclosure, the umbilical cord blood (UCB) sample was taken from umbilical vein within 3 minutes (approximately between 2-5 minutes) of the baby's delivery. The sample collection was performed when the placenta was still in utero. The procedure for UCB sample collection involved clamping the umbilical cord after it stops pulsating to collect the cord blood sample. Strict aseptic techniques were used for the cleaning of umbilical cord with alcohol followed by povidone iodine. After cleansing step, the umbilical vein was pierced and cord blood was collected in the standard blood collection bag. The 80-100 ml cord blood was shaken gently in order to freely mix with 22 ml (approximately) citrate phosphate dextrose adenine-1 (CPDA-1), that is already present as anticoagulant in the collection bag (Terumo Penpol cord Blood collection Bag). Post-mixing the cord blood sample with anticoagulant, the UCB sample was immediately transported from maternity units to the processing center in cold chain as the samples were to be processed for the isolation of leucocyte rich concentrate within 48 hours from the time of collection.

Before proceeding for the isolation process, 3 ml of collected cord blood was obtained through one of the spikes by inserting a coupler for microbial testing and total nucleated cell count (TNCC). The microbial tests showed that the UCB sample collected in the present experient was free from any microbial contamination. The maternal blood (birthing mothers blood) test showed negative/non reactive for infectious diseases related to Human Immunodeficiency Virus (HIV), Hepatitis C Virus (HCV), Hepatitis B surface antigen (HBsAg) and Syphilis. The total nucleated cell count for the preprocessed UCB sample was found to be ≥1-2×10⁸ per unit. Further, the pre-processing cord blood selection included In-utero collection of at least 40 ml of cord blood volume from volunteers having full-term deliveries, i.e., >36 weeks.

### Example 2

### Isolation of Leucocyte Rich Concentrate

The method for the isolation and enrichment of CD34+, CD56+, and CD3+ cell population includes the sedimentation step as explained in the present example. The sedimentation allows separation of RBCs from the umbilical cord blood (UCB) samples as obtained in Example 1 of the present disclosure, wherein the sedimentation reagents including hydoxy ethyl starch (HES), gelatin and chitosan were used for the isolation of leucocyte rich concentrate. Table 1 of the present disclosure showed the initial differential cell count of cord blood samples as obtained in the Example 1.

TABLE 1

| Blood cell type | Amount |
|---|---|
| Red blood cells (×10⁶/μL) | 3890 ± 760 |
| Hematocrit (%) | 32.4 ± 4.1 |
| MCv (fL) | 112.4 ± 8.2 |
| MCH (g/dl) | 34.2 ± 1.9 |
| Hemoglobin (g/dl) | 12.5 ± 1.8 |
| Platelets (×10⁹/μL) | 165 ± 20.1 |
| White blood cells (×10⁶/μL) | 5.9 ± 4.1 |
| Lymphocytes (%) | 48.5 ± 12.4 |
| Monocytes (%) | 11.8 ± 5.7 |

For the sedimentation purpose, each of three cord blood unit samples (Sample 1, Sample 2, and Sample 3) as obtained in the previous example was split into three equal parts for treatment with 6% HES, 3% gelatin, and 75% chitosan respectively. After disinfecting the inlet of sedimentation reagent (SDR) bottle with alcohol swab, of afore-mentioned sedimentation reagents were taken from the SDR bottle in a syringe and injected into the umbilical cord blood (UCB) collection bag aseptically. The cord blood and sedimention reagent are present in a ratio range of 2:1 wherein 2 ml of cord blood and 1 ml of sedimentation reagent is used. Further, the syringe used for transfer of respective sedimentation reagents were discarded after use. The UCB collection bag was then placed on the rocker (Major Science Co. Ltd.) for 5 minutes to facilitate proper mixing of sedimentation reagent (SDR) with umbilical cord blood. Followed by the mixing in the previous step, the UCB collection bag was transferred into the Biosafety cabinet. The UCB processing bag set was removed by opening the peelable package, and then visually inspected to ensure that all the clamps were closed and the bag was intact. In case the outer bag is found tampered, the UCB processing bag set is eliminated for use to avoid contamination. The UCB Bag-1 spike was then inserted into the remaining spike port of UCB Collection bag using aseptic technique. The droplets sticking on the wall of UCB Collection bag were brought down carefully to the bottom by squeezing the bag between forefinger and thumb, followed by placing the UCB Collection bag for sedimentation on the sedimentation rack for 35 (between 30-90) minutes under undisturbed conditions. After the completion of sedimentation time, the UCB Collection bag was placed on the plasma expressor to separate leucocyte rich plasma. Table 2 of the present disclosure depicts the initial whole blood cell count (103), post-process leucocyte count of cord blood unit (10⁸), cell recovery (%), initial hematocrit amount (%), post-process hematocrit amount (%), and cell viability (%) for all three cord blood unit samples (Sample 1, Sample 2, and Sample 3) after treatment with sedimentation reagents. The clamp between UCB Collection bag was set open to express the leukocyte rich plasma from the UCB collection bag to UCB Bag-1. The flow of plasma was stopped by closing the clamp between UCB Collection bag and UCB Bag-1 before the settled red cells from UCB Collection bag were just about to enter UCB Bag-1, ensuring no movement of RBC's into the UCB Bag-1. The tubing between UCB Bag-1 to UCB Collection bag was then sealed further to detach the collection bag from UCB Bag-1. The UCB bag set containing the leukocyte rich plasma was subjected to centrifugation at 450 g/1260 RPM for 10 minutes for Thermofisher centrifuge at 20° C. Followed by the centrifugation, the UCB bag set was taken out from centrifuge carefully and, the UCB Bag-1 was placed on the Auto Volume Expresser (Cellbios Pvt. Ltd.) in Biosafety Cabinet. The clamp between the UCB Bag-1 and UCB Bag-2 were set open to express supernatant plasma into UCB Bag-2, post which the tubing between UCB Bag-1 and UCB Bag-2 was sealed in order to detach UCB bag-2 from UCB Bag-1. The residual cord blood plasma in UCB Bag-2 as saved for the extraction of plasma protein. For the assessment of post-process sterility under quality control, 10 ml each of residual RBC and plasma were from detached collection bag and UCB bag 2 respectively in 50 ml falcon tube. The concentrated nucleated cells obtained post centrifugation, i.e., buffy coat in UCB Bag-1 was mixed by gentle mixing. All the cell suspension obtained in the form of leucocyte concentrate were then transferred to falcon tubes with help of syringe for further RBC lysis.

TABLE 2

| QC | SAMPLE 1 | | | SAMPLE 2 | | | SAMPLE 3 | | |
|---|---|---|---|---|---|---|---|---|---|
| Parameter | Gelatin-1 | HES-1 | Chitosan-1 | Gelatin-2 | HES-2 | Chitosan-2 | Gelatin-3 | HES-3 | Chitosan-3 |
| INITIAL WHOLE | 2.9 | 1.9 | 1.3 | 2.12 | 3 | 3.4 | 1.64 | 1.08 | 1.56 |
| BLOOD COUNT (10⁸) | | | | | | | | | |
| POST PROCESS | 2.7 | 1.86 | 1.29 | 1.91 | 2.9 | 3.35 | 1.54 | 1.01 | 1.54 |
| LEUCOCYTE | | | | | | | | | |
| COUNT OF CBU (10⁸) | | | | | | | | | |
| RECOVERY (%) | 93.1 | 97.89 | 99.23 | 90.09 | 96.66 | 98.52 | 93.9 | 93.51 | 98.71 |
| Initial HCT (%) | 27 | | | 30 | | | 25 | | |
| Post process HCT (%) | 0.7 | 1 | 0.5 | 0.8 | 1.3 | 0.7 | 0.9 | 1.4 | 0.5 |
| Viability (%) | 96.5 | 96.3 | 97 | 95.1 | 95.5 | 95 | 97.2 | 96.9 | 97 |

As per the Table 2, the post-process leucocyte count of cord blood samples showed appreciable figures against the initial whole blood cell count in the sample, signifying the efficiency of the sedimentation process as performed herein in terms of cell loss. In fact, above 90% cell recovery was observed in all the three cord blood samples treated with sedimentation reagents respectively, wherein chitosan showed maximum cell recovery percentage in all the three samples, i.e., 99.23%, 98.52%, and 98.71% in Sample 1, Sample 2, and Sample 3 respectively. The evident decrease in the hematocrit percentage in the post-processed samples against the initial hematocrit percentage highlighted effective removal of RBC's through the sedimentation process as performed herein. Additionally, the results as per Table 2 showed ≥95% leucocyte cell viability of the recovered leucocyte cell as obtained after the double sedimentation process.

Further, the effect of different combinations including gelatin-hydroxyethyl starch (1:1); hydoxyethyl starch-chitosan (1:1); chitosan-gelatin (1:1); and gelatin-hydroxyethyl starch-chitosan (1:1:1) were studied in the sedimentation step as performed in the experiment conducted in the present example. The results for initial whole blood cell count (10³), post-process leucocyte count of cord blood unit (10⁸), cell recovery (%), initial hematocrit amount (%), post-process hematocrit amount (%), and cell viability (%) after the use of said combinations as sedimentation reagents for all three cord blood unit samples (Sample 01, Sample 02, and Sample 03) respectively to obtain leucocyte rich plasma were depicted in Table 3. The abbreviations, GE, HE, and CH in Table 3 corresponds to gelatin, hydroxyethyl starch and chitosan respectively.

TABLE 3

| | SAMPLE 01 | | | | SAMPLE 02 | | | | SAMPLE 03 | | | |
|---|---|---|---|---|---|---|---|---|---|---|---|---|
| QC | GE:HE | HE:CH | CH:GE | GE:HE:CH | GE:HE | HE:CH | CH:GE | GE0:HE:CH | GE:HE | HE:CH | CH:GE | GE:HE:CH |
| Parameter | (1:1) | (1:1) | (1:1) | (1:1:1) | (1:1) | (1:1) | (1:1) | (1:1:1) | (1:1) | (1:1) | (1:1) | (1:1:1) |
| INITIAL | 3.1 | 2.61 | 3.20 | 2.9 | 2.7 | 3.3 | 2.8 | 3.11 | 2.7 | 2.52 | 3.0 | 2.42 |
| WHOLE | | | | | | | | | | | | |
| BLOOD | | | | | | | | | | | | |
| COUNT (10⁸) | | | | | | | | | | | | |
| POST | 2.91 | 2.5 | 3.1 | 2.85 | 2.55 | 3.1 | 2.68 | 2.98 | 2.58 | 2.38 | 2.9 | 2.35 |
| PROCESS | | | | | | | | | | | | |
| LEUCOCYTE | | | | | | | | | | | | |
| COUNT | | | | | | | | | | | | |
| OF CBU(10⁸) | | | | | | | | | | | | |
| RECOVERY(%) | 93.87 | 95.78 | 96.87 | 98.27 | 94.44 | 93.93 | 95.71 | 95.81 | 95.55 | 94.44 | 96.66 | 97.10 |
| Initial HCT(%) | 28 | | | | 26 | | | | 29 | | | |
| Post process | 0.8 | 0.9 | 0.5 | 0.6 | 0.9 | 1.1 | 0.8 | 0.9 | 0.8 | 1 | 0.7 | 0.8 |
| HCT(%) | | | | | | | | | | | | |
| Viability(%) | 97.5 | 95.6 | 98 | 97.3 | 96.1 | 97.5 | 96 | 97.3 | 96.8 | 96 | 98 | 97.3 |

The results in Table 3 showed marginal difference in initial whole blood cell count and post-sedimentation leucocyte count of cord blood samples 1, 2, and 3 respectively, therefore it could be inferred that the sedimentation step as performed herein led to efficient selective sedimentation of blood cells like RBC's, thus separating out leucocyte rich concentrate for further processing. The sedimentation reagents including gelatin-hydroxyethyl starch (1:1); hydoxyethyl starch-chitosan (1:1); and chitosan-gelatin (1:1) showed significant percentage of cell recovery and cell viability among the leaucocyte rich cord blood samples obtained after the sedimentation step, however, gelatin-hydroxyethyl starch-chitosan (1:1:1) combination depicted unexpectedly high percentage of cell recovery for all three cord blood unit samples (Sample 1, Sample 2, and Sample 3) respectively. In general, higher percentage of cell recovery was observed in case of sedimentation reagent including chitosan in combination with gelatin/hydroxyethyl starch or both. Additionally, the post sedimentation hematocrit percentage observed in all three cord blood unit samples was least in case of samples treated with gelatin-hydroxyethyl starch-chitosan (1:1:1) combination against gelatin-hydroxyethyl starch (1:1); hydoxyethyl starch-chitosan (1:1); and chitosan-gelatin (1:1) combinations respectively. Therefore, it could be inferred that sedimentation regent having gelatin, hydroxyethyl starch, and chitosan in a ratio of 1:1:1 showed synergistic and efficient depletion of unwanted blood cells as compared to their individual results depicted in Table 2. The cord blood samples thus obtained after sedimentation with gelatin-hydroxyethyl starch-chitosan (1:1:1) combination having more than 1.3×10⁵ CD34+/CD56+/CD3+ cell count and more than 70% total nucleated cell count and CD34+/CD56+/CD3+ cell viability were taken forward for further processing as per the present disclosure.

### Example 3

### Cord Blood and Maternal Blood Plasma Protein Extraction

The leucocyte concentrate as obtained in Example 2 was then further carried forward for RBC depletion procedure with ammonium chloride or in combination with cord blood and maternal blood plasma proteins. The present example pertains to the process followed for the extraction of plasma proteins from cord blood and maternal blood plasma to be used in RBC depletion in cord blood samples.

The residual cord blood plasma in UCB Bag-1 was used for the extraction of cord blood plasma protein. The cord blood plasma was stored at 2-8° C., till sterility result confirmation was received. Further, maternal whole blood sample from the same donor as that of the cord blood was tested for the absence of Infectious diseases such as HIV, HCV, HBsAg and Syphilis. Maternal blood plasma was obtained from maternal whole blood sample by centrifugation at 2000 rpm for 5 minutes in the form of supernatant. Once after the confirmation received for the absence of any infectious agents in cord blood plasma and whole maternal blood sample, the maternal blood plasma was mixed with cord blood plasma for use in the preparation of proteins. In order to facilitate the precipitation of plasma derived proteins, saturated ammonium sulphate or ethanol was added to the mixture of cord blood and maternal blood plasma in ratio of 1:1 ((Cord blood plasma+maternal blood plasma):(saturated ammonium sulphate or ethanol)). The plasma mix with saturated ammonium sulphate or ethanol was allowed to stand for 10 minutes for the precipitation of plasma proteins, which was then subjected to centrifugation at 3000 (3000-5000) rpm for 5 minutes in order to settle down the protein precipitate in the form of pellet. Post-centrifugation, the supernatant was discarded and around 4-5 ml of water or Saline or Dulbecco's phosphate buffer saline was used to dissolve the pellet. The dissolved pellet mixture was then again centrifuged at 11,000 (11,000-14,000) rpm for 5 minutes, and the supernatant was collected in separate tube for use in preparation of powdered form of plasma derived proteins. The collected supernatant was allowed to lyophilize under vacuum conditions using vacuum lyophilizer, followed by the assessment for total plasma protein extracted. The plasma derived proteins as obtained in the present example were then stored for use in further RBC depletion of leucocyte rich concentrate as obtained in Example 2 of the present disclosure.

### Example 4

### Red Blood Cell Depletion

The leucocyte rich cord blood samples as obtained in Example 2 of the present disclosure, were equally divided in five sets for further RBC depletion through different methods, namely Method-I, Method-II, Method-III, Method-IV, and Method V, wherein the aforementioned methods involved the subjection of cord blood samples in five individual sets to ammonium chloride lysis buffer; ammonium chloride lysis buffer and feotal bovine serum (FBS) combination; ammonium chloride lysis buffer and human serum albumin (HSA) combination; ammonium chloride lysis buffer and blood plasma derived protein (Example 3) combination; and no treatment respectively. All the five different sample sets after RBC depletion with aforementioned methods were subjected to cell enrichment step.

All the five leucocyte rich cord blood sets post RBC depletion with respective methods were analyzed for the percentage expression of the early apoptotic cells, late apoptotic cells, live cells, and dead cells as shown in Table 4 of the present disclosure. As shown in Table 4, each of the five sets were further divided into three samples (Sample 01, Sample 02, Sample 03) for apoptotic studies. Cell apoptosis studies in the present disclosure were performed based on flow cytometry with Annexin-V marker post RBC depletion step.

TABLE 4

| | | | | | | | | | | RBC LYSIS WITH | | | STARTING | | |
|---|---|---|---|---|---|---|---|---|---|---|---|---|---|---|---|
| | RBC LYSIS WITH | | | RBC LYSIS WITH | | | RBC LYSIS WITH | | | AMMONIUM | | | SAMPLE | | |
| | AMMONIUM | | | AMMONIUM | | | AMMONIUM | | | CHLORIDE + CB AND | | | (Leucocyte | | |
| | CHLORIDE | | | CHLORIDE + FBS | | | CHLORIDE + HSA | | | MATERNAL PLASMA | | | concentrate) | | |
| Percentage | (METHOD-I) | | | (METHOD-II) | | | (METHOD-III) | | | PROTEIN (METHOD-IV) | | | (METHOD-V) | | |
| expression | Sam- | Sam- | Sam- | Sam- | Sam- | Sam- | Sam- | Sam- | Sam- | Sam- | Sam- | Sam- | Sam- | Sam- | Sam- |
| (%) | ple | ple | ple | ple | ple | ple | ple | ple | ple | ple | ple | ple | ple | ple | ple |
| EARLY | 1.7 | 2.3 | 1.9 | 1.3 | 2 | 2.8 | 1.8 | 1.9 | 2.1 | 0.7 | 1.7 | 1.5 | 0.8 | 1.2 | 1.3 |
| APOPTOTIC | | | | | | | | | | | | | | | |
| CELLS | | | | | | | | | | | | | | | |
| LATE | 4.1 | 4.2 | 3 | 2.5 | 2.2 | 2.6 | 2.5 | 2.7 | 2.7 | 0.3 | 1.3 | 1.1 | 0.2 | 1 | 0.8 |
| APOPTOTIC | | | | | | | | | | | | | | | |
| CELLS | | | | | | | | | | | | | | | |
| LIVE | 89.1 | 88.1 | 89 | 93.1 | 92.9 | 91 | 92.8 | 91.6 | 92.1 | 97.9 | 95.1 | 95.3 | 98.1 | 96.8 | 96.7 |
| CELLS | | | | | | | | | | | | | | | |
| DEAD | 5.1 | 5.4 | 6.1 | 3.1 | 2.9 | 3.6 | 2.9 | 3.8 | 3.1 | 1.1 | 1.9 | 2.1 | 0.9 | 1 | 1.2 |
| CELLS | | | | | | | | | | | | | | | |

Further to the results as reflected in Table 4 for the apoptotic and cell viability studies, the early apoptotic cell expression was observed highest in case of cord blood treated with ammonium chloride and FBS (Method-II), which was then followed by samples treated with ammonium chloride and HSA (Method-III) and then the samples treated with ammonium chloride (Method-I). The cord blood samples treated with ammonium chloride and blood plasma derived protein combination (Method-1V), and leucocyte concentrate (Method V) were observed to have very less early apoptotic cell expression in comparison to that observed in Method-I, Method-II, and Method-III respectively. Whereas, the late apoptotic cell expression was observed highest in case of cord blood samples treated with ammonium chloride (Method-I), followed by cord blood samples treated with ammonium chloride and FBS (Method-II), and ammonium chloride and HSA (Method-III). Similar to the early apoptotic cell expression cord blood samples treated with ammonium chloride and blood plasma derived protein combination (Method-1V), and leucocyte concentrate (Method V) were observed to have very less late apoptotic cell expression in comparison to rest methods. In the samples treated with ammonium chloride (Method-I), highest number of dead cell expression with least live cell expression (<90%) was observed. The cord blood samples treated with ammonium chloride and FBS (Method-II), and ammonium chloride and HSA (Method-III) were observed to have comparatively higher live cell expression than in Method-I, whereas untreated cord blood samples (Method-V) were observed to have highest live cell expression and lowest dead cell expression followed by the cord blood samples treated with ammonium chloride and blood plasma derived protein combination (Method-IV). Therefore, among all the test methods performed for RBC depletion in cord blood samples as per Table 4, the cord blood samples treated with ammonium chloride and blood plasma derived protein combination (Method-IV) showed significantly appreciable results in terms of least apoptotic cell expression and maximum live cell expression with least dead cell expression.

Further to results obtained as per Table 4, the comparative effects on cell viability of cells in cord samples treated with different concentration of ammonium chloride (Method-I), and ammonium chloride and blood plasma derived protein combination (Method-IV) were studied. The 1× concentration as depicted in the Table 4 for ammonium chloride (Method-I) has 8.26 g of ammonium chloride in 1 litre of distilled water, and ammonium chloride and blood plasma derived protein combination (Method-IV) out of which, 4 ml of ammonium chloride and 200 ug of proteins was used for 1 ml of leucocyte concentrate of cord blood. The results as obtained from the said comparative study are reflected in Table 5 of the present disclosure, wherein with the increasing concentration of ammonium chloride i.e., 1λ, 2×, 4×, 6×, 8×, and 10× showed 90%, 89.1%, 85%, 79%, 77%, and 59% of live cell expression respectively after RBC lysis in cord blood samples. Whereas, the decrease in the live cell expression in cord blood samples treated with the increasing concentration of ammonium chloride and blood plasma derived protein combination (Method-1V) was comparatively negligible as compared to the trend observed in case of samples treated with Method-I, as 1×, 2×, 4×, 6×, 8×, and 10× of ammonium chloride and blood plasma derived protein combination showed 97.5%, 97%, 97.2%, 96.1%, 93.5%, and 93.1% live cells expression in lysed cord blood samples. Hence, it could be inferred that, the plasma derived protein in combination with ammonium chloride (Method-1V) as disclosed herein helped to efficiently retain cell viability even after further RBC depletion in cord blood samples.

TABLE 5

| | | | | | | | | RBC LYSIS WITH | | | | | |
|---|---|---|---|---|---|---|---|---|---|---|---|---|---|
| | | RBC LYSIS WITH | | | | | | AMMONIUM CHLORIDE | | | | | |
| | | AMMONIUM CHLORIDE | | | | | | AND PROTEIN | | | | | |
| | Starting | (Method-I) | | | | | | COMBINATION (Method-IV) | | | | | |
| | sample | 1X | 2X | 4X | 6X | 8X | 10X | 1X | 2X | 4X | 6X | 8X | 10X |
| Percentage | 98 | 90 | 89.1 | 85 | 79 | 77 | 59 | 97.5 | 97 | 97.2 | 96.1 | 93.5 | 93.1 |
| expression | | | | | | | | | | | | | |
| of live cells (%) | | | | | | | | | | | | | |

Post-analyzing the effect of using blood plasma derived protein combination with ammonium chloride (Method-IV) in RBC depletion of cord blood samples, the comparative effects on early apoptotic cells, late apoptotic cells, live cells, and dead cells expression of cord blood samples treated with different blood derived proteins including Peroxiredoxin-1, Heat shock cognate 71 kDa protein isoform 2, Isoform 3 of N-alpha-acetyltransferase 60, Isoform 3 of Serine/threonine-protein kinase Chkl, Peroxiredoxin-1 and Heat shock cognate 71 kDa protein isoform 2 cobination, and all blood derived protein combination with ammonium chloride respectively were studied.

Human peroxiredoxin 1 (hPrx1) is a member of the peroxiredoxin family, which detoxifies peroxide substrates and has been implicated in numerous biological processes, including cell growth, proliferation, differentiation, apoptosis, and redox signaling. Thiol-specific peroxidase that catalyzes the reduction of hydrogen peroxide and organic hydroperoxides to water and alcohols, respectively. Plays a role in cell protection against oxidative stress by detoxifying peroxides.

Heat shock proteins (hsp) are highly conserved proteins that can be constitutively expressed and function to fold proteins into mature tertiary structures, or they can be induced and function to restore partially damaged proteins under conditions of stress. The Heat shock 70 kDa protein 8 also known as heat shock cognate 71 kDa protein or Hsc70 or Hsp73 facilitates the proper folding of newly translated and misfolded proteins, as well as stabilize or degrade mutant proteins. As a member of the heat shock protein 70 family and a chaperone protein, it facilitates the proper folding of newly translated and misfolded proteins, as well as stabilize or degrade mutant proteins. Its functions contribute to biological processes including signal transduction, apoptosis, autophagy. The underlying ability of Hsps to maintain cell survival correlates with an inhibition of caspase activation and apoptosis that can, but does not always, depend upon their chaperoning activities. Several signaling cascades involved in the regulation of key elements within the apoptotic cascade are also subject to modulation by Hsps, including those involving JNK, NFκB and AKT. The coordinated activities of the Hsps thus modulate multiple events within apoptotic pathways to help sustain cell survival following damaging stimuli.

The results for the present study are reflected in Table 6, wherein cord blood samples treated with ammonium chloride and all blood (cord blood+maternal blood) derived proteins showed relatively higher live cell expression (≥95%) and lower early & late apoptotic cell expression than the cord blood samples treated with ammonium chloride in combination with individual blood derived proteins including, Peroxiredoxin-1, Heat shock cognate 71 kDa protein isoform 2, Isoform 3 of N-alpha-acetyltransferase 60, and Isoform 3 of Serine/threonine-protein kinase Chkl respectively. Unlike the trends observed for early apoptotic cells, late apoptotic cells, live cells, and dead cells expression in cord blood samples treated with ammonium chloride in combination with individual blood derived proteins, significantly higher live cell expression (>95%) and lower early & late apoptotic cell expression was observed in case of cord blood samples treated with ammonium chloride in combination with Peroxiredoxin-1 and Heat shock cognate 71 kDa protein isoform 2. Further, similar trends for early & late apoptotic cell expression was observed in case of cord blood samples treated with ammonium chloride and all blood derived proteins combination, and ammonium chloride and Peroxiredoxin-1—Heat shock cognate 71 kDa protein isoform 2 combination, however, higher higher live cell expression (>95%) was observed in the latter treatment for RBC lysis. Therefore, it could be inferred that, not all blood derived proteins in combination with ammonium chloride would deliver the appreciable results in terms of cell viability and cell apoptosis aspects. Only certain protein combination like, Peroxiredoxin-1 and Heat shock cognate 71 kDa protein isoform 2 with ammonium chloride has the capability to deliver unexpected synergistic RBC depletion in cord blood samples. Further, the RBC depleted samples as obtained from the treatment of cord blood samples with ammonium chloride in combination with Peroxiredoxin-1 and Heat shock cognate 71 kDa protein isoform 2 were carried forward for target cell enrichment.

TABLE 6

| | | | | | | | | | | RBC LYSIS WITH | |
|---|---|---|---|---|---|---|---|---|---|---|---|
| | | | | | | | | | | AMMONIUM | |
| | | | | | | | | | | CHLORIDE and | |
| | | | | RBCs lysis with | | | | | | Heat shock | |
| | | | | ammonium | | | RBC LYSIS WITH | | | cognate | |
| | | | | chloride and | | | AMMONIUM | | | 71 kDa | |
| Percentage | STARTING | | | CB + MB | | | CHLORIDE and | | | protein | |
| expression | SAMPLE (LC) | | | derive protein | | | Peroxiredoxin-1 | | | isofrom 2 | |
| (%) | Sample 01 | Sample02 | Sample 03 | Sample 01 | Sample02 | Sample 03 | Sample 01 | Sample 02 | Sample 03 | Sample 01 | Sample 02 |
| Early | 0.8 | 1.2 | 1.3 | 0.7 | 1.7 | 1.5 | 0.9 | 1.9 | 1.7 | 0.7 | 1.8 |
| apoptotic | | | | | | | | | | | |
| cells | | | | | | | | | | | |
| Late | 0.2 | 1 | 0.8 | 0.3 | 1.3 | 1.1 | 1 | 1.5 | 2.1 | 2 | 1.9 |
| apoptotic | | | | | | | | | | | |
| cells | | | | | | | | | | | |
| Live cells | 98.1 | 96.8 | 96.7 | 97.8 | 95 | 95.2 | 96.1 | 94.5 | 93.2 | 95.3 | 93.9 |
| Dead cells | 0.9 | 1 | 1.2 | 1.2 | 1.8 | 2.3 | 2 | 2.1 | 2.9 | 2 | 2.4 |

| | | | | | | | | RBC LYSIS | | |
|---|---|---|---|---|---|---|---|---|---|---|
| | | | | | RBC LYSIS | | | WITH | | |
| | | RBC LYSIS | | | WITH | | | AMMONIUM | | |
| | | WITH | | | AMMONIUM | | | CHLORIDE and | | |
| | RBC LYSIS | AMMONIUM | | | CHLORIDE and | | | Peroxiredoxin-1 | | |
| | WITH | CHLORIDE and | | | Isoform 3 | | | 1 - Heat shocked | | |
| | AMMONIUM | Isoform 3 | | | of Serine/ | | | coagnate 71 | | |
| | CHLORIDE and | of N-alpha- | | | threonine- | | | kDa protein | | |
| Percentage | Heat shock | acetyltransferase | | | protein kinase | | | isoform 2 | | |
| expression | cognate | 60 | | | Chkl | | | combination | | |
| (%) | Sample 03 | Sample 01 | Sample 02 | Sample 03 | Sample 01 | Sample 02 | Sample 03 | Sample 01 | Sample 02 | Sample 03 |
| Early | 1.7 | 1.9 | 2 | 2.3 | 2.8 | 2.1 | 2.3 | 0.7 | 1.7 | 1.5 |
| apoptotic | | | | | | | | | | |
| cells | | | | | | | | | | |
| Late | 2.2 | 5.2 | 3.1 | 4.1 | 3.8 | 6.3 | 5.3 | 0.3 | 1.0 | 1.1 |
| apoptotic | | | | | | | | | | |
| cells | | | | | | | | | | |
| Live cells | 94.1 | 88 | 89.8 | 90 | 89.9 | 87.8 | 88.2 | 97.9 | 95.1 | 95.3 |
| Dead cells | 2 | 4.9 | 5.1 | 3.6 | 3.5 | 3.8 | 4.2 | 1.1 | 1.9 | 2.1 |

The method performed for the depletion of RBCs as per the present example was carried out through five different methods, wherein Method-V was leucocyte rich cord blood sample as obtained in Example 2 (untreated control). Method-I involved the treatment of 0.5 ml leucocyte rich cord blood sample with 2 ml RBC lysis buffer (1% ammonium chloride lysing buffer) to remove traces of RBCs in a ratio of 1:4 (leucocyte rich cord blood:lysing buffer) followed by incubation in ice bucket for 10 minutes. The leucocyte rich cord blood and lysing buffer mix was diluted with 10 ml of phosphate buffer saline (PBS) followed by centrifugation at 2000 rpm for 10 minutes. The pellet as obtained after centrifugation was washed twice with buffer. Similar process as mentioned for Method-I was followed for Method-II, Method-III, and Method IV, wherein the lysing buffer for Method-II, Method-III, and Method IV were 1% ammonium chloride lysing buffer in ωmbinationation with human serum albumin (HSA), fetal bovine serum (FBS), and Peroxiredoxin-1 Protein—Heat shock cognate 71 kDa protein isoform 2 protein combination, respectively. After RBC lysis as performed via Method-I, Method-II, Method-III, and Method IV, the pellets obtained after washing was resuspended in 300 μL of buffer (Phosphate buffer saline (PBS)+EDTA+HSA) per 10⁸ total cells for use in cell viability and cell apoptosis analysis as performed in the present example.

### Example 5

### Marker-Specific Cell Enrichment

The present example pertains to the marker specific hematopoietic stem cell (HSC), NK cell, and T cell enrichment, wherein the RBC depleted samples as obtained in Example 4 using five different methods were separately enriched for the afore-mentioned cell types. The method as performed herein for the enrichment of hematopoietic stem cells (HSC's), NK cells, and T cells involves the use of fluorescence-activated cell sorting (FACS), that provides a method for sorting a heterogeneous mixture of blood cells based upon the specific light scattering and fluorescent characteristics of each cell.

(a) HSC enrichment: The RBC depleted cord blood samples were enriched for HSC cells having positive surface markers selected from the consisting of CD34+, CD133+, CD38−, and negative surface markers selected from the consisting of CD3+, CD19+, CD56+. The results as obtained for percentage expression of the aforementioned markers on HSCs in RBC depleted samples obtained by five different methods as described in Example 4 are depicted in Table 7 of the present disclosure.

TABLE 7

| | | ENRICHMENT | | | | | | | |
|---|---|---|---|---|---|---|---|---|---|
| | | AFTER | | | | | | | |
| | | RBC LYSIS | | | ENRICHMENT | | | ENRICHMENT | |
| | | WITH | | | AFTER | | | AFTER | |
| | | AMMONIUM | | | AMMONIUM | | | AMMONIUM | |
| | | CHLORIDE | | | CHLORIDE + FBS | | | CHLORIDE + HSA | |
| CD | | (METHOD-I) | | | (METHOD-II) | | | (METHOD-III) | |
| MARKER | % | Sample | Sample | Sample | Sample | Sample | Sample | Sample | Sample |
| FUNCTION | EXPRESSION | 01 | 02 | 03 | 01 | 02 | 03 | 01 | 02 |
| HSC | CD34+, | 82.72 | 84.02 | 87.02 | 92 | 93.1 | 90.1 | 91.9 | 92.3 |
| POSITIVE | CD133+, | | | | | | | | |
| | CD38− | | | | | | | | |
| HSC | CD3+ | 0.79 | 0.81 | 0.95 | 0.9 | 1.6 | 1.1 | 1.1 | 0.8 |
| | CD19+ | 3.10 | 2.11 | 1.79 | 1.6 | 2.9 | 1.6 | 1.3 | 2 |
| | CD56+ | 2.34 | 3.11 | 2.98 | 2.3 | 3.1 | 2.8 | 3.2 | 2.9 |

| | | | ENRICHMENT | | | | | |
|---|---|---|---|---|---|---|---|---|
| | | | AFTER | | | | | |
| | | | RBC LYSIS | | | | | |
| | | ENRICHMENT | WITH | | | | | |
| | | AFTER | AMMONIUM | | | | | |
| | | AMMONIUM | CHLORIDE + | | | ENRICHMENT NO | | |
| | | CHLORIDE + HSA | PROTEIN | | | RBC LYSIS | | |
| CD | | (METHOD-III) | (METHOD-IV) | | | (METHOD-V) | | |
| MARKER | % | Sample | Sample | Sample | Sample | Sample | Sample | Sample |
| FUNCTION | EXPRESSION | 03 | 01 | 02 | 03 | 01 | 02 | 03 |
| HSC | CD34+, | 89.9 | 99.12 | 97.8 | 98.0 | Column | | |
| POSITIVE | CD133+, | | | | | block | | |
| | CD38− | | | | | | | |
| HSC | CD3+ | 2.1 | 0.03 | 0.01 | 0.0 | | | |
| | CD19+ | 0.02 | 0.02 | 0.03 | 0.02 | | | |
| | CD56+ | 1.8 | 0.08 | 0.04 | 0.07 | | | |

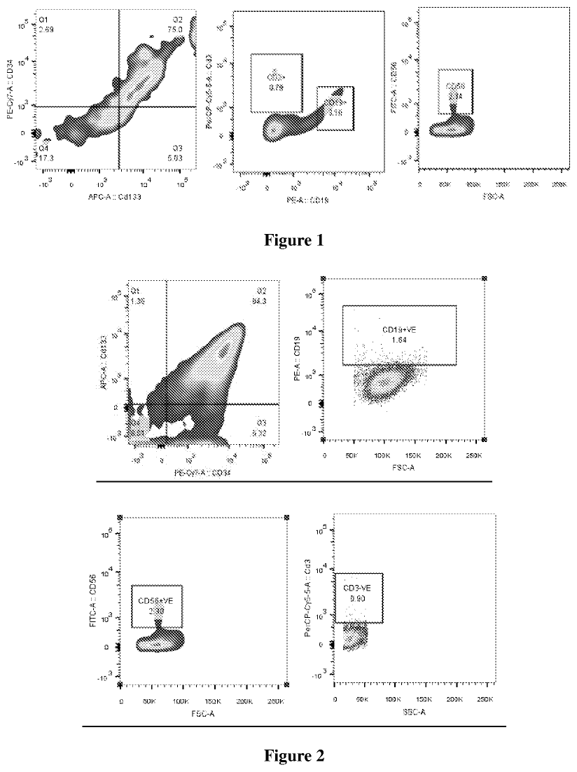

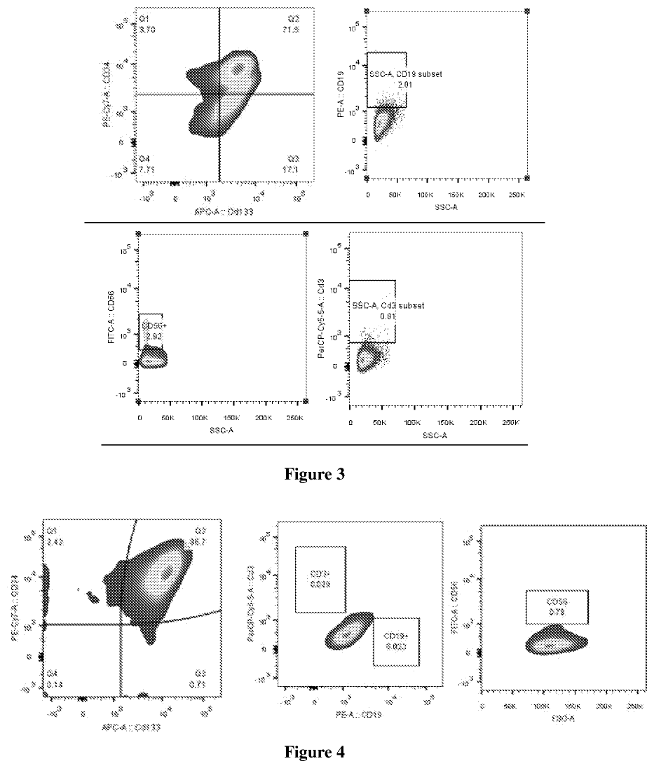

As per the results obtained for fluorescence-activated cell sorting (FACS) for marker-specific enrichment of HSC's are reflected in Table 7, the percentage expression of HSCs with positive CD markers (CD34+, CD133+, and CD38−) was observed least (<87%) in case of RBC depleted cord blood samples treated with Method-I among Method-I to IV. There were no results obtained for marker-specific enrichment in case of Method-V wherein no RBC lysis was performed, as the RBC's present in the cord blood samples led to column blockage. FIG. 1 depicts the fluorescence-activated cell sorting (FACS) results as obtained in case of HSC's enriched with Method-I (Sample 01), wherein 82.72% of CD34+ and CD133+ cell purity was achieved. The percentage expression of HSCs with positive CD markers in case of RBC depleted cord blood samples treated with Method-II was marginally higher than as observed in case of cord blood samples treated with Method-III. FIG. 2 depicts the fluorescence-activated cell sorting (FACS) results as obtained in case of HSC's enriched with Method-II (Sample 01), wherein 92% of CD34+ and CD133+ cell purity was achieved. However, significantly high percentage expression (>97.8%) of HSCs with positive CD markers in case of RBC depleted cord blood samples treated with ammonium chloride and synergistic protein combination (Method-IV) was observed. Therefore, with the least expression of negative CD markers, including CD3+ (<0.03%), CD19+ (<0.03%), and CD56+ (<0.08%) observed in case of RBC depleted cord blood samples treated with ammonium chloride and synergistic protein combination, Method-IV was considered as most efficient pathway for RBC depletion among all the five methods as performed in the Example 4 of the present disclosure. FIGS. 3 and 4 depicts the fluorescence-activated cell sorting (FACS) results as obtained in case of HSC's enriched with Method-III (Sample 01) and Method IV (Sample 01), wherein 92.3% and 99.12% of CD34+ and CD133+ cell purity respectively was achieved.

(b) Natural-killer (NK) cell enrichment: For the NK cell enrichment, the cells in RBC depleted cord blood samples having positive surface markers selected from the consisting of CD56+, and CD3−, and negative surface markers selected from the consisting of CD34+, and CD19+ respectively were separated. Table 8 of the present disclosure depicts the results as obtained for percentage expression of the aforementioned markers on NK cells in RBC depleted samples obtained by five different methods as described in Example 4.

TABLE 8

| | | ENRICHMENT AFTER | | | | | | |
|---|---|---|---|---|---|---|---|---|
| | | RBC LYSIS | | | | | | |
| | | WITH | | | ENRICHMENT AFTER | | | ENRICHMENT AFTER |
| | | AMMONIUM | | | AMMONIUM | | | AMMONIUM |
| CD | | CHLORIDE | | | CHLORIDE + FBS | | | CHLORIDE + HSA |
| MARKER | % | (METHOD-I) | | | (METHOD-II) | | | (METHOD-III) |
| FUNCTION | EXPRESSION | Sample 01 | Sample 02 | Sample 03 | Sample 01 | Sample 02 | Sample 03 | Sample 01 |
| NK | CD56+ | 81.8 | 85 | 88.3 | 91.7 | 90.1 | 93.1 | 92 |
| POSITIVE | CD3− | | | | | | | |
| NK | CD34+ | 3.39 | 2.91 | 4.11 | 1.90 | 2.11 | 1..51 | 2.53 |
| NEGATIVE | CD19+ | 4.29 | 3.20 | 3.5 | 2.21 | 2.63 | 3.1 | 2.43 |
| MARKER | | | | | | | | |

| | | | | ENRICHMENT AFTER | | | | |
|---|---|---|---|---|---|---|---|---|
| | | | | RBC LYSIS | | | | |
| | | | | WITH | | | | |
| | | ENRICHMENT AFTER | | AMMONIUM | | | | |
| | | AMMONIUM | | CHLORIDE + | | | ENRICHMENT NO | |
| CD | | CHLORIDE + HSA | | PROTEIN | | | RBC LYSIS | |
| MARKER | % | (METHOD-III) | | (METHOD-IV) | | | (METHOD-V) | |
| FUNCTION | EXPRESSION | Sample 02 | Sample 03 | Sample 01 | Sample 02 | Sample 03 | Sample 01 | Sample 02 |
| NK | CD56+ | 91.3 | 90 | 97.4 | 96.8 | 98.5 | Column | |
| POSITIVE | CD3− | | | | | | block | |
| NK | CD34+ | 1.70 | 1.90 | 0.88 | 0.91 | 0..61 | | |
| NEGATIVE | CD19+ | 2.91 | 3.11 | 1.32 | 1.93 | 1.30 | | |
| MARKER | | | | | | | | |

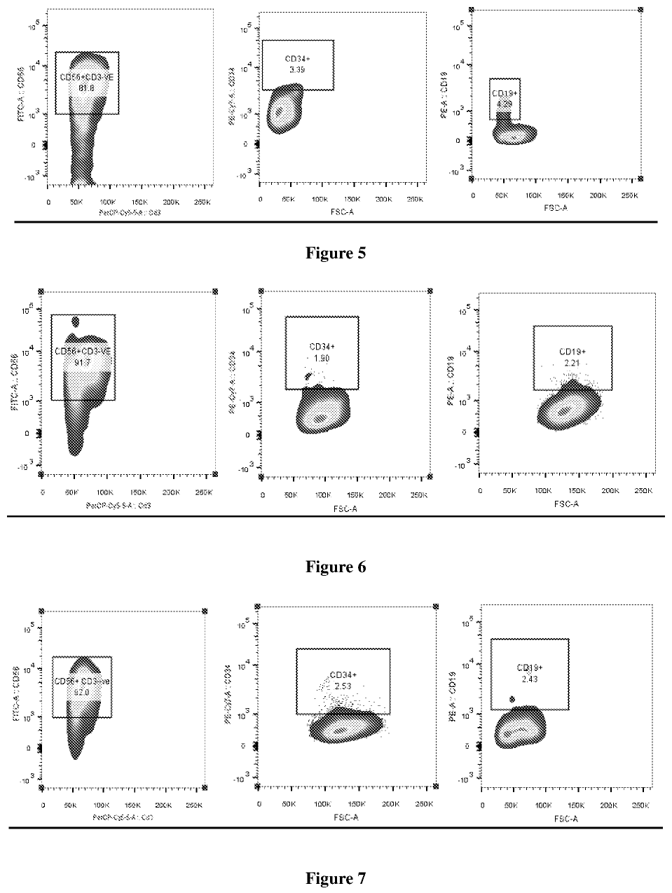

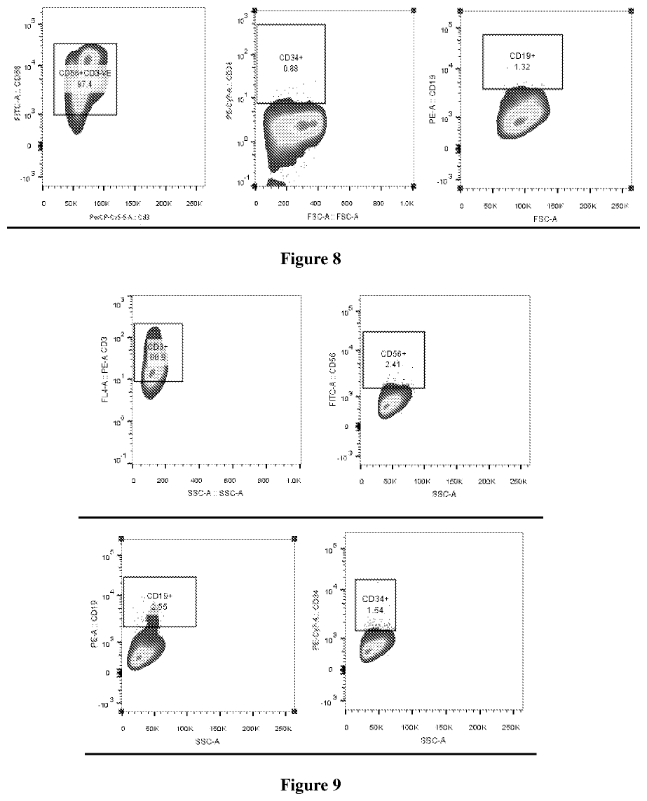

Table 8 shows the results obtained for marker-specific enrichment of NK cells, wherein the percentage expression of NK cells with positive CD markers (CD56+, and CD3−) was observed least (<88.3%) in case of RBC depleted cord blood samples treated with Method-I among Method-I to IV. Similar to the HSC enrichment, there were no results obtained for marker-specific enrichment of NK cells in case of Method-V. FIG. 5 depicts the fluorescence-activated cell sorting (FACS) results as obtained in case of NK cells enriched with Method-I (Sample 01), wherein 81.8% of CD56+ and CD3− cell purity was achieved. The percentage expression of NK cells with positive CD markers in case of RBC depleted cord blood samples treated with Method-II was marginally higher than as observed in case of cord blood samples treated with Method-III. FIG. 6 depicts the fluorescence-activated cell sorting (FACS) results as obtained in case of NK cells enriched with Method-II (Sample 01), wherein 91.7% of CD56+ and CD3− cell purity was achieved. However, significantly high percentage expression (>96.8%) of NK cells with positive CD markers in case of RBC depleted cord blood samples treated with ammonium chloride and synergistic protein combination (Method-IV) was observed. Additionally, the least expression of negative CD markers in case of samples treated with ammonium chloride and synergistic protein combination, Method-IV was considered to be the most efficient method among all five methods, I, II, III, IV, and V as depicted in Table 4 of the present disclosure. The FIGS. 7 and 8 depicts the images of fluorescence-activated cell sorting (FACS) results as obtained in case of NK cells enriched with Method-III, and IV respectively, wherein 92% and 94% of CD56+ and CD3− cell purity was achieved.

(c) T cell enrichment: T cell enrichment in RBC depleted cord blood samples was carried out by selecting the cells having CD3+ positive surface markers and negative surface markers selected from the consisting of CD34+, CD56+, and CD19+ respectively. Table 9 of the present disclosure depicts the results as obtained for percentage expression of the aforementioned markers on T cells in RBC depleted samples obtained by five different methods as described in Example 4.

TABLE 9

| | | ENRICHMENT AFTER | | | | | | | |
|---|---|---|---|---|---|---|---|---|---|
| | | RBC LYSIS | | | | | | | |
| | | WITH | | | ENRICHMENT AFTER | | | ENRICHMENT AFTER | |
| | | AMMONIUM | | | AMMONIUM | | | AMMONIUM | |
| CD | | CHLORIDE | | | CHLORIDE + FBS | | | CHLORIDE + HSA | |
| MARKER | % | (METHOD-I) | | | (METHOD-II) | | | (METHOD-III) | |
| FUNCTION | EXPRESSION | Sample 01 | Sample 02 | Sample 03 | Sample 01 | Sample 02 | Sample 03 | Sample 01 | Sample 02 |
| T CELL | CD3+ | 88.9 | 87.6 | 86.1 | 91.4 | 90.2 | 89.9 | 92.6 | 90.3 |
| T CELL | CD34+ | 1.64 | 2.90 | 3.11 | 1.83 | 2.12 | 2.70 | 2.25 | 1.92 |
| NEGATIVE | CD56+ | 2.41 | 3.11 | 2.92 | 2.68 | 1.93 | 2.91 | 1.98 | 2.11 |
| | CD19+ | 2.55 | 3.88 | 3.22 | 3.07 | 2.31 | 3.3 | 3.68 | 2.67 |

| | | | ENRICHMENT AFTER | | | | | |
|---|---|---|---|---|---|---|---|---|
| | | | RBC LYSIS | | | | | |
| | | | WITH | | | | | |
| | | ENRICHMENT AFTER | AMMONIUM | | | | | |
| | | AMMONIUM | CHLORIDE + | | | ENRICHMENT NO | | |
| CD | | CHLORIDE + HSA | PROTEIN | | | RBC LYSIS | | |
| MARKER | % | (METHOD-III) | (METHOD-IV) | | | (METHOD-V) | | |
| FUNCTION | EXPRESSION | Sample 03 | Sample 01 | Sample 02 | Sample 03 | Sample 01 | Sample 02 | Sample 03 |
| T CELL | CD3+ | 91.2 | 96 | 98.2 | 97.6 | Column | | |
| T CELL | CD34+ | 2 | 0.94 | 1.9 | 1.72 | block | | |
| NEGATIVE | CD56+ | 2.40 | 1.32 | 0.99 | 1.63 | | | |
| | CD19+ | 2.11 | 0.84 | 1.8 | 1.51 | | | |

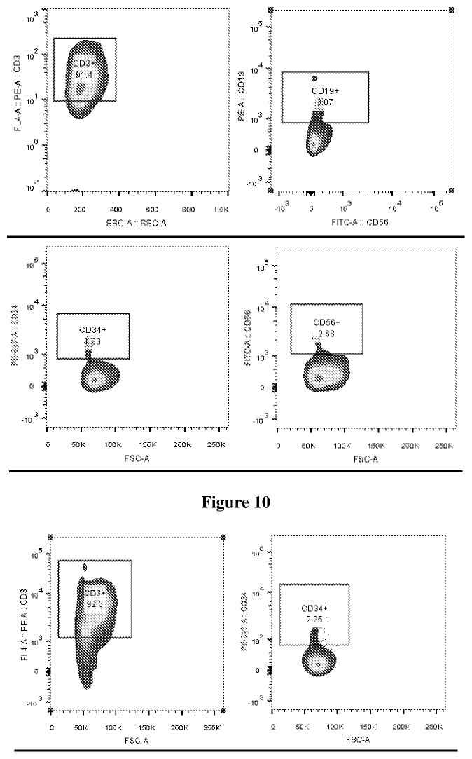

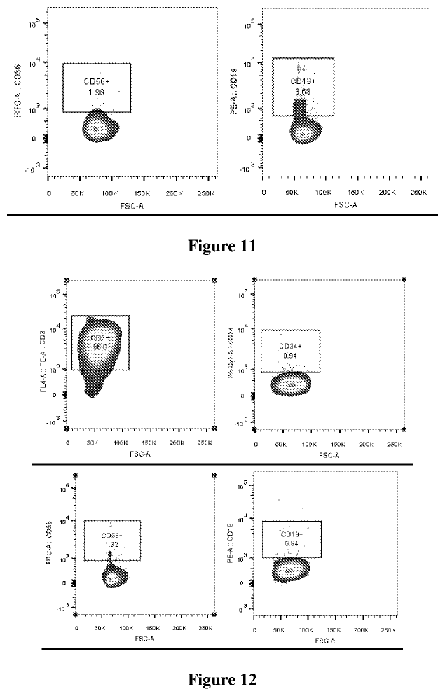

Table 9 shows the results obtained for marker-specific enrichment of T cells, wherein the percentage expression of T cells with positive CD3+ marker was observed least (<88.9%) in case of RBC depleted cord blood samples treated with Method-I among Method-I to IV. Similar to the HSC and NK cell enrichment results, there were no results obtained for marker-specific enrichment of T cells in case of Method-V. FIGS. 9 and 10 depicts the fluorescence-activated cell sorting (FACS) results as obtained in case of T cells enriched with Method-I (Sample 01) and Method-II (Sample 01) respectively, wherein 88.9% and 91.4% of CD3+ cell purity was achieved. The percentage expression of T cells with positive CD markers in case of RBC depleted cord blood samples treated with Method-III was marginally higher than as observed in case of cord blood samples treated with Method-II. However, significantly high percentage expression (>96%) of T cells with positive CD markers in case of RBC depleted cord blood samples treated with ammonium chloride and synergistic protein combination (Method-IV) was observed. Further, the least expression of negative CD markers (CD34+, CD56+, and CD19+) in case of samples treated with ammonium chloride and synergistic protein combination, Method-IV was considered to be the most efficient method among all five methods as performed in Example 4 of the present disclosure. FIGS. 11 and 12 depicts the fluorescence-activated cell sorting (FACS) results as obtained in case of T cells enriched with Method-III (Sample 01) and Method-IV (Sample 01) respectively, wherein 92.6% and 96% of CD3+ cell purity was achieved.

Therefore, the marker expression results obtained for positive and negative markers in the cell enrichment step performed in the present example establishes that, Method-IV was the most signicant method for RBC depletion in the method disclosed herein to obtain HSC, NK and T cell population in accordance with the present disclosure. Moreover, isolated HSC, CD3 and CD56 cells can be cultured ex-vivo through various methods and used as a ready to use allogenic or autologous therapy for the different malignant and non malignant diseases.

Overall, it could be summarised from the results as obtained in all the examples provided herein that the present disclosure discloses a significantly efficient method for the isolation of HSC's, NK and T cell population from the umbilical cord blood. The use of hydroxyethyl starch, gelatin, chitosan and combinations thereof in the sedimentation reagent showed effective sedimentation in order to obtain leucocyte rich plasma as per the present method. Further, double sedimentation step as performed herein enhances the removal of unwanted blood cell population at starting stages. Further, the maternal and cord-blood derived protein combination along with ammonium chloride showed synergistic results in further depletion of RBC's in leucocyte rich cord blood obtained after double-sedimentation. The fluorescence-activated cell sorting (FACS) as performed herein helped for marker-specific cell enrichment in the RBC depleted cell population to obtain HSC (CD34+, CD133+, and CD38−), NK (CD56+, and CD3−), and T (CD3+) cell population. The enriched cell population was then further subjected to cell expansion to be used in various therapeutic purposes.

### Advantages of the Present Disclosure

Unlike the conventional method available in the field of art, the method as disclosed herein for obtaining enriched hematopoietic stem cells, natural killer cells and T cells focusses on achieving higher cell expansion capacity, higher recovery of surface marker-specific cell population, higher cell viability, and minimizing cell apoptosis in the expanded cell population. Further, the use of umbilical cord blood as starting material provides non-limiting availability of starting material, lower chances of severe graft-versus-host disease (GVHD) without compromising graft-versus-leukemia effects, less stringent criteria for human leukocyte antigen (HLA) matching, lower risk of viral transmission and the absence of risk to donors. The conventional methods of HSC's isolation and enrichment are generally known to compromise either with the cell count or cell purification aspects depending upon the purpose-specific use, however, the present method as disclosed herein involves double-sedimentation step to maximise removal of unwanted blood cells to arrive at leucocyte rich concentrate, followed by RBC depletion using ammonium chloride in combination with maternal and cord blood derived proteins to further elevate the fraction of desired hematopoietic stem cells, natural killer cells and T cells population in the cord blood samples. The marker-specific cell enrichment and expansion provides ready-to-use cell population for therapeutic or related purposes. Further, the use of cord and maternal plasma proteins combination in addition to ammonium chloride for RBCs lysis as disclosed herein provides protective effect on cells viability even at higher concentration of ammonium chloride used for the said purpose. The nullifying effect of cord and maternal plasma proteins combination against the free-radical and oxidative stress generated by ammonium chloride in RBC lysis maximises the cell recovery of the targeted HSCs, NK and T cell population. The immunomagnetic cell separation step helps in further enrichment of target cells, thereby enhancing the purity of the desired cell population. Overall, the present disclosure provides an effective solution to the existing problems in the present field of art to obtain HSCs, NK and T cell population for therapeutic purposes.

I/We claim:

1. A method for obtaining enriched haemopoietic stem cells, natural killer cells and T cells, wherein the method comprising:
a) separating leucocytes from umbilical cord blood using a sedimentation reagent to obtain leucocyte rich plasma, wherein the sedimentation buffer comprises at least one of the reagents selected from the group consisting of hydroxy ethyl starch, gelatin, chitosan, and combinations thereof;
b) adding red blood cell lysis buffer to the leucocyte rich plasma to obtain leucocyte concentrate, wherein the red blood cell lysis buffer comprises ammonium chloride and a protein combination comprising at least one protein derived from umbilical cord blood and maternal blood, wherein the leucocyte concentrate is of red blood cells;
c) incubating the leucocyte concentrate with CD34⁺, CD133⁺ and CD38⁻ immunomagnetic beads for obtaining hematopoietic stem cells;
d) incubating the leucocyte concentrate with CD56⁺ and CD3⁻ immunomagnetic beads for obtaining natural killer cells; and
e) incubating the leucocyte concentrate with CD3+ immunomagnetic beads for obtaining T cells.

2. The method as claimed in claim 1, wherein the hydroxy ethyl starch is in the concentration range of 4-12% with the molecular weight in the range of 130 kDa-500 kDa, the gelatin is in the concentration range of 3-6% with the molecular weight in the range of 30-60 kDa, and the chitosan with the molecular weight in the range of 50-247 kDa.

3. The method as claimed in claim 1, wherein the red blood cell lysis buffer is obtained by a method comprising:
a) obtaining a plasma from an umbilical cord blood;
b) obtaining a plasma from a maternal blood;
c) mixing the plasma from umbilical cord blood and the plasma from maternal blood to obtain a first mixture;
d) adding saturated ammonium sulphate to the first mixture in ratio of 1:1 followed by centrifugation at 3000-5000 rpm for 5 minutes to obtain the protein precipitate in the form of pellet;
e) suspending the pellet in water followed by centrifugation at 11,000-14,000 rpm to obtain an enriched plasma; and
f) adding ammonium chloride to the enriched plasma to obtain the red blood cell lysis buffer, wherein said lysis buffer comprises ammonium chloride and a protein combination, wherein said protein combination comprises antioxidative protein, serine/threonine protein kinase, transmembrane protein and heat shock proteins.

4. The method as claimed in claim 3, wherein the antioxidative protein is peroxiredoxin-1 protein and heat shock proteins are heat shock cognate 71 kDa protein isoform 2.

5. The method as claimed in claim 1, wherein the hematopoietic stem cells, natural killer cells, and T cells have a purity of at least 90%.

6. The method as claimed in claim 1, wherein the hematopoietic stem cells, natural killer cells, and T cells obtained are in the range of 10⁵-10⁶ cells.

7. An enriched hematopoietic stem cells (HSC) obtained by the method as claimed in anyone of the claims 1-6.

8. An enriched natural killer cells obtained by the method as claimed in anyone of the claims 1-6.

9. An enriched T cells obtained by the method as claimed in anyone of the claims 1-6.

10. A composition comprising: (a) an enriched hematopoietic stem cells (HSC) as claimed in claim 7, having positive and negative surface markers; and (b) pharmaceutically acceptable excipients selected from HSA, DMEM or combinations thereof.

11. The composition as claimed in claim 10, wherein said positive surface markers for hematopoietic stem cells are selected from the group consisting of CD34⁺, CD133⁺ and CD38⁻, and the negative markers selected from the group consisting of CD2, CD3, CD11b, CD14, CD15, CD16, CD19, CD56, CD123, and CD235a.

12. A composition comprising: (a) an enriched natural killer cells as claimed in claim 8, wherein natural killer cells are having positive and negative surface markers; and (b) pharmaceutically acceptable excipients selected from HSA, DMEM or combinations thereof.

13. The composition as claimed in claim 12, wherein said positive surface markers for natural killer cells are selected from the group consisting of CD56⁺, and CD3⁻ and negative markers selected from the group consisting of CD34, and CD19.

14. A composition comprising: (a) an enriched T cells as claimed in claim 9, wherein T cells are having positive and negative surface markers; and (b) pharmaceutically acceptable excipients selected from HSA, DMEM or combinations thereof.

15. The composition as claimed in claim 14, wherein said positive surface markers for T cells is CD3+ and said negative markers are selected from CD56, CD34, and CD19.

16. The composition as claimed in anyone of the claims 10-15 for use in a subject in need.

17. An isolated cell population enriched for CD34⁺, CD133⁺ and CD38⁻ haematopoietic stem cells.

18. An isolated cell population enriched for CD56⁺, and CD3⁻ Natural killer cells.

19. An isolated cell population enriched for CD3+ T cells.

20. A method of treating diseases selected from the group consisting of graft versus host disease, malignant disease, and non-malignant disease, said method comprising administering a therapeutic amount of the composition claimed in any of the claims 10-15, to the subject.
